(19) United States
(12) Patent Application Publication
(10) Pub. No.: US 2019/0256896 A1
(43) Pub. Date: Aug. 22, 2019

(54) MEASURING METHOD FOR LOW CONCENTRATION NUCLEIC ACID SAMPLE

(71) Applicant: Quark Biosciences, Inc., Hsinchu County (TW)

(72) Inventors: Cheng-Wey Wei, Hsinchu County (TW); Chang-Wei Huang, Hsinchu County (TW); Chung-Fan Chiou, Hsinchu County (TW)

(73) Assignee: Quark Biosciences, Inc., Hsinchu County (TW)

(21) Appl. No.: 15/976,860

(22) Filed: May 10, 2018

(30) Foreign Application Priority Data
Feb. 21, 2018 (TW) 107105731

(51) Int. Cl. C12Q 1/6816

(52) U.S. Cl. CPC C12Q 1/6816

(57) ABSTRACT

A measuring method for low concentration nucleic acid sample is provided. The measuring method is a nucleic acid measuring method for samples of low nucleic acid concentration range in a qPCR experiment, so as to extend the linear dynamic range of experimental detection and increase detecting sensitivity by correcting the original Cq value. The method includes providing a test plate with a plurality of reaction wells or holes, so as to perform qPCR reaction on the nucleic acid samples. Next, performing the adjustment step based on the positive well measurement value derived from the number of positive wells, so as to correct the Cq value of qPCR reaction to the expected value of linear range.

### CROSS-REFERENCE TO RELATED APPLICATIONS

This application claims the priority benefit of Taiwan Patent Application No. 107105731, filed on Feb. 21, 2018. The entirety of the above-mentioned patent application is hereby incorporated by reference herein and made a part of this specification.

### BACKGROUND OF THE INVENTION

### Field of the Invention

The invention relates to a measuring method, and more particularly, to a measuring method for low concentration nucleic acid sample.

### Description of Related Art

Currently, most high-density array format qPCR systems have the issue of low sensitivity. When low concentration nucleic acid sample is tested on high-density array format qPCR system, the amount of input nucleic acid is not enough to be distributed into all reaction wells. The linear range of the Cq values (quantification cycle) is affected by the single copy number in each reaction well. As a result, the Cq value of the qPCR reaction converges to a constant value, so the resolution and detection power for low concentration nucleic acid sample are poor. Moreover, multiple nucleic acid targets within a sample may have a wide concentration range, which is particularly common in clinical samples. If a lower concentration nucleic acid target exists, the aforementioned issue is very likely to occur.

For instance, FIG. 1 shows the relationship between the average Cq value and log of cDNA concentration known in the art. Referring to FIG. 1, when the average Cq value is between 8 and 26, a linear relationship (Cq stable zone) can be maintained between the average Cq value and the logarithm of cDNA concentration (log of concentration is between 4 and 10), and the dynamic range is about 6 logs. However, in the low concentration range, the sample distributed in each reaction well is less than a single copy in average and the Cq value converges to a constant, which interrupts the linearity. At this point, the average copy number in each well is lower (such as less than 1 copy or unevenly distributed), so the reliability of the Cq value is also negatively affected, and the limit of quantitation (LoQ) is usually between 5 and 50 copy numbers for each reaction well. A qPCR detection can only be successfully performed when a linear relationship is presented, otherwise the issue of poor accuracy may occur.

In prior art, the aforementioned issue is mainly solved via PCR pre-amplification, wherein the sample concentration is increased to 10⁴ times or more via 14 to 20 PCR amplification cycles, so as to move the Cq values from un-stable zone to stable zone, which presents a linear relationship. However, a clinical sample usually contains multiple nucleic acid targets, so the pre-amplification method may not amplify different nucleic acid targets equally in the same reaction, which causes that the final measurement result may not represents the concentration before amplification.

Based on the above observations, improving the stability and sensitivity and extending the dynamic range of real-time quantitative polymerase chain reaction (qPCR) by correcting the Cq value for low concentration nucleic acid sample is an important issue, especially for samples which contain multiple nucleic acid targets with different concentrations.

### SUMMARY OF THE INVENTION

The invention provides a measuring method for a nucleic acid sample, which is able to improve the stability and sensitivity of qPCR and extend the dynamic range. The measuring method is suitable for samples which contain different nucleic acid targets having a variety of concentration ranges.

The measuring method for a nucleic acid sample of the invention includes the following steps. A test plate having a plurality of reaction wells is provided to perform the qPCR reaction on the nucleic acid sample. Next, when the positive well measurement value obtained by the number of positive reaction wells represents the sample concentration is less than a constant, an adjustment step is performed to correct the original Cq value.

In an embodiment of the invention, the adjustment step includes the following steps. A qPCR reaction efficiency is first obtained. For instance, a graph is drawn by using the nucleic acid template concentration as the horizontal axis and the original Cq value as the vertical axis, and the slope is obtained. After the slope is able to be converted into qPCR efficiency, the slope is multiplied by the logarithm of positive well measurement value and the original Cq value is added, so as to obtain the corrected Cq value.

In an embodiment of the invention, 64 reaction wells of a test plate are used in the qPCR reaction according to this measuring method.

In an embodiment of the invention, the nucleic acid sample contains more than one kind of nucleic acid target, and the nucleic acid targets have different concentration ranges.

In an embodiment of the invention, after using the 64 reaction wells and the adjustment step, the dynamic range is increased to 9 logs.

In an embodiment of the invention, the correlation coefficient R² between the nucleic acid template concentration and the corrected Cq value is 0.98 or more.

In an embodiment of the invention, dividing the number of positive reaction wells by the number of all reaction wells to obtain a ratio of positive reaction wells to all reaction wells, and then plugging the ratio into the Poisson distribution to obtain an average sample copy number of each reaction well, wherein the average sample copy number of each reaction well is the positive well measurement value.

In an embodiment of the invention, when the average sample copy number of each reaction well (which is called positive well measurement value) is less than 1, the adjustment step is performed to correct the original Cq value.

In an embodiment of the invention, the positive well measurement value can be the ratio obtained by dividing the number of positive reaction wells by the number of all reaction wells.

In an embodiment of the invention, when the ratio obtained by dividing the number of positive reaction wells by the number of all reaction wells (which is called positive well measurement value) is less than 95%, the adjustment step is performed to correct the original Cq value.

Based on the above, the invention provides a measuring method for a nucleic acid sample, which is able to adjust the Cq value of a nucleic acid target with lower concentration, so as to improve qPCR sensitivity and extend the dynamic range. More specifically, the dynamic range can be increased from 6 logs (Cq value between 6 and 25) to 9 logs (Cq value between 6 and 35) by using the method of this invention.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a graph showing the relationship between the average Cq value and the logarithm of cDNA concentration known in the art.

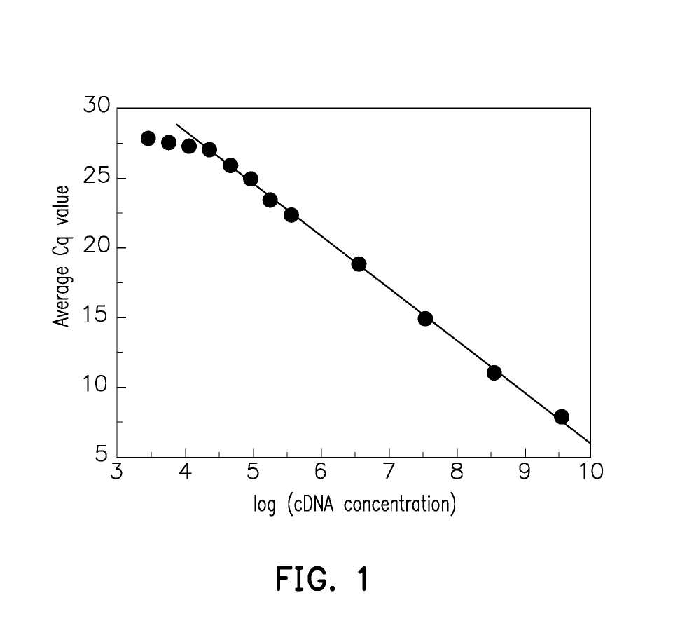

FIG. 2 is a mechanism schematic of the measuring method for a nucleic acid sample according to the invention.

FIG. 3 is a graph showing the relationship between average Cq value and sample concentration of a pUC57 cDNA nucleic acid sample.

### DESCRIPTION OF THE EMBODIMENTS

The invention provides a measuring method for a nucleic acid sample. In the following, the terms used in the specification are defined first.

“qPCR” or “real-time quantitative PCR” (real-time quantitative polymerase chain reaction) refers to an experimental method of using PCR to amplify and quantify target DNA at the same time. Quantification was performed using a plurality of measuring chemical substances (including fluorescent dye of SYBR® green or fluorescent reporter oligonucleotide probe of Taqman probe, for instance), and real-time quantification is performed with the amplified DNA accumulated in the reaction after every amplification cycle.

“cDNA” (complementary DNA) refers to complementary DNA generated by performing reverse transcription on an RNA template using reverse transcriptase.

“Cq value” is the number of amplification cycles when fluorescence intensity starts to be significantly increased in a qPCR operation process.

“Sample” refers to a nucleic acid sample to be tested. For instance, the sample can be a nucleic acid segment (including DNA or RNA, etc.) extracted from a source such as blood, tissue, or saliva. “Template” refers to DNA, RNA or micro RNA chain having a specific sequence, which is also called biological marker and can be detected by a qPCR reaction.

“Positive reaction wells” refers to reaction wells showing positive results in a qPCR reaction, and “negative reaction wells” refers to reaction wells showing negative results in a qPCR reaction.

“Test plate having a plurality of reaction wells” refers to a carrier plate having a plurality of reaction wells, wherein each reaction well is used for a qPCR reaction.

The invention provides a measuring method for a nucleic acid sample, including first providing a test plate having a plurality of reaction wells to perform a qPCR reaction on the nucleic acid sample, and the nucleic acid sample may contain one or more kind of nucleic acid target. When the nucleic acid sample contains more than one kind of nucleic acid target, the concentration range of each nucleic acid target may be different, and may even be significantly different. The reaction wells in the test plate are individually distributed to measure different types of nucleic acid templates having a variety of concentration ranges. In the present embodiment, a qPCR reaction is performed using 64 reaction wells of a test plate, for instance.

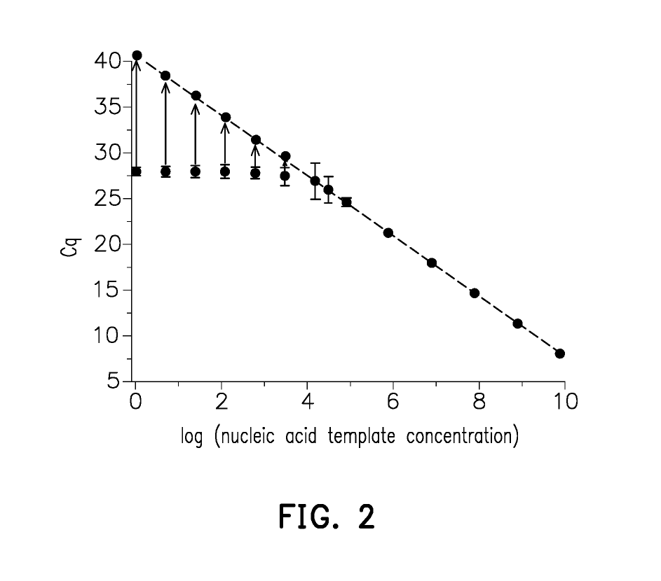

FIG. 2 is a mechanism schematic of the measuring method for a nucleic acid sample according to the invention. As shown in FIG. 2, the main mechanism of the invention is to adjust the Cq value converging to a constant in a low concentration range into a linear relationship. More specifically, the positive well measurement value is obtained according to the number of positive reaction wells in the invention. When this measurement value represents the sample is in low concentration, an adjustment step is performed to correct the original Cq value.

The positive well measurement value of the invention includes but is not limited to the following two kinds of measurement value. The first is the average sample copy number of each reaction well obtained according to the Poisson distribution. More particularly, the number of positive reaction wells is divided by the number of all reaction wells to obtain a ratio of positive reaction wells to all reaction wells, and then the ratio is plugged into the Poisson distribution, so as to obtain the average sample copy number of each reaction well as the positive well measurement value. When the average sample copy number of each reaction well (which is called positive well measurement value) is less than 1, an adjustment step is performed to correct the original Cq value. The second is the ratio of positive reaction wells to all reaction wells. More particularly, the ratio obtained by dividing the number of positive reaction wells by the number of all reaction wells is a kind of positive well measurement value. When the ratio of positive reaction wells to all reaction wells is less than 95%, an adjustment step is performed to correct the original Cq value.

In the present embodiment, the positive well measurement value can be the average sample copy number of each reaction well as described in the following: the number of positive reaction wells is divided by the number of all reaction wells to obtain a ratio of positive reaction wells to all reaction wells, and the ratio is plugged into the Poisson distribution to obtain the average sample copy number of each reaction well. The Poisson distribution is as follows:

λ=−ln(1−k/n)

wherein λ represents the average sample copy number of each reaction well, n represents the number of all reaction wells, and k represents the number of positive reaction wells.

When the average sample copy number of each reaction well (which is called positive well measurement value) is less than 1, an adjustment step is performed to correct the original Cq value, so that the original Cq value converges to be a constant in a low concentration range is corrected back to a linear relationship. For instance, the adjustment step may include: drawing a graph by using the nucleic acid template concentration as the horizontal axis and the original Cq value as the vertical axis and obtaining a slope (this slope is related to PCR efficiency), and then multiplying the slope by the logarithm of positive well measurement value and adding the original Cq value, so as to obtain the corrected Cq value. As a result, the original Cq value converges to be a constant in the low concentration range can be adjusted to a corrected Cq value, so as to approach the expected linear relationship shown in FIG. 2.

In the present embodiment, the positive well measurement value can be the ratio of positive reaction wells to all reaction wells, wherein the ratio is obtained by dividing the number of positive reaction wells by the number of all reaction wells, as described below:

μ=k/n

wherein n represents the number of all reaction wells, and k represents the number of positive reaction wells.

When the ratio of positive reaction wells to all reaction wells is less than 95%, an adjustment step is performed to correct the original Cq value in the manner provided above.

After using the 64 reaction wells of a test plate and the adjustment step, the dynamic range can be increased from 6 logs (Cq value between 6 and 25) to 9 logs (Cq value between 6 and 35) via the measuring method for the nucleic acid sample of the invention. Moreover, the correlation coefficient R² between the nucleic acid template concentration and the corrected Cq value is 0.99 or more.

In the following, each of the design methods is described in detail via the experimental examples. However, the following experimental examples are not intended to limit the invention.

### Experimental Example

To prove that the measuring method for a nucleic acid sample provided in the invention is able to correct the Cq value and increase the sensitivity and accuracy of qPCR, an experimental example is provided below, including Example 1 with a pUC57 cDNA nucleic acid sample and Example 2 with a human reference RNA nucleic acid sample.

### Cq Value and Correlation Coefficient R² Evaluation (Example 1)

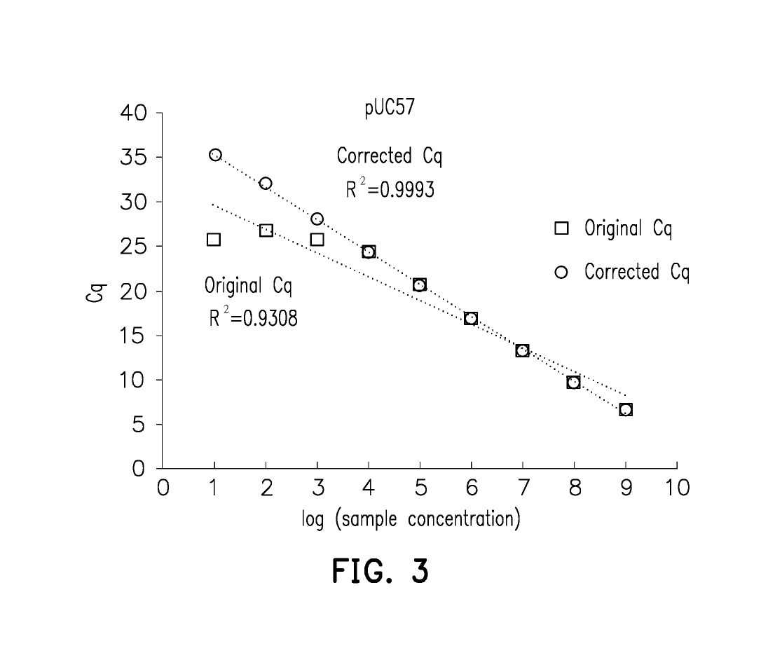

Serial dilution is performed on a pUC57 cDNA nucleic acid sample, and the sample concentration, original Cq value, and corrected Cq value of the nucleic acid targets are measured and shown in Table 1 and FIG. 3. As shown in FIG. 3, the Cq of the first 6 measuring points is highly related to the sample concentrations (the logarithm of sample concentrations is 9, 8, 7, 6, and 5, with a Cq between 5 and 25). For the 3 lowest measuring points (the logarithm of sample concentrations is 3, 2, and 1) in FIG. 3, the average concentration of each reaction well is less than one copy number, so the Cq value converges to a fixed value (about 26 in the present example) and the correlation coefficient is not high (R² value is 0.93). After the Cq of the last three measuring points are corrected using the method of the invention, R² is increased to 0.99. In the present example, the dynamic range is increased from 6 logs (Cq between 5 and 25) to 9 logs (Cq between 5 and 35).

TABLE 1

| | | Corrected Cq value | Corrected Cq value |
|---|---|---|---|
| | | (Corrected from | (Corrected from the |
| The logarithm | | the average sample | ratio of positive |
| of Sample | Original | copy number of | reaction wells to all |
| concentration | Cq value | each reaction well) | reaction wells) |
| 9 | 6.67 | 6.67 | 6.67 |
| 8 | 9.71 | 9.71 | 9.71 |
| 7 | 13.23 | 13.23 | 13.23 |
| 6 | 16.93 | 16.93 | 16.93 |
| 5 | 20.70 | 20.70 | 20.70 |
| 4 | 24.40 | 24.40 | 24.40 |
| 3 | 25.85 | 28.19 (corrected) | 28.31 (corrected) |
| 2 | 26.79 | 32.20 (corrected) | 32.24 (corrected) |
| 1 | 25.95 | 35.33 (corrected) | 35.32 (corrected) |

### Cq Value, Correlation Coefficient R², and PCR Efficiency Evaluations (Example 2)

Serial dilution is performed on human reference RNA nucleic acid sample, and qPCR reaction is performed on a test plate having a plurality of reaction wells. The nucleic sample contains nucleic acid targets of a plurality of different concentration ranges such as Beta-Actin, HER2, PD-1, and c-Met. The original Cq value, correlation coefficient R², and PCR efficiency of each nucleic acid target are measured, and the measurement results are shown in Table 2.

In Table 2, in the case of PD-1, the Cq value at 300 ng is 24.44, and the Cq value is increased by 2.91 to be 27.35 when diluted 4 times to 75 ng, However, for 4-time dilutions after 9.38 ng, the Cq value only increased slightly (such as increased only by 0.05 from 30.06 to 30.11). Since the second half of Cq values are not increased in proportion, the correlation coefficient between all of the dilutions and the Cq values is only 0.919. Next, the ratio of positive reaction wells to all reaction wells (the number of positive reaction wells/the number of all reaction wells) was plugged into the Poisson distribution to obtain the average copy number of each reaction well, which is the positive well measurement value. Afterwards, when the average copy number of each reaction well (which is called positive well measurement value) is less than 1, an adjustment step is performed to correct the original Cq value. The corrected Cq value, correlation coefficient R², and PCR efficiency are shown in Table 3. Moreover, the ratio obtained by dividing the number of positive reaction wells by the number of all reaction wells is used as the positive well measurement value. When the ratio is less than 95%, an adjustment step is performed to correct the original Cq value. The corrected Cq value, correlation coefficient R², and PCR efficiency are shown in Table 4.

TABLE 2

| Original | | | | | | | Correlation | PCR |
|---|---|---|---|---|---|---|---|---|
| Cq value | 300 ng | 75 ng | 18.75 ng | 9.38 ng | 4.69 ng | 2.34 ng | coefficient R² | efficency |
| Beta Actin | 18.39 | 20.94 | 23.28 | 24.43 | 25.52 | 26.62 | 0.999 | 81% |
| | (84/84) | (84/84) | (84/84) | (84/84) | (84/84) | (84/84) | | |
| | c/w > 3 | c/w > 3 | c/w > 3 | c/w > 3 | c/w > 3 | c/w > 3 | | |
| HER2 | 26.11 | 28.42 | 29.61 | 29.71 | 30.18 | 30.43 | 0.915 | 223% |
| | (84/84) | (84/84) | (34/84) | (18/84) | (10/84) | (5/84) | | |
| | c/w > 3 | c/w > 3 | c/w = 0.52 | c/w = 0.24 | c/w = 0.13 | c/w = 0.06 | | |
| PD-1 | 24.44 | 27.35 | 28.66 | 30.06 | 30.11 | 30.14 | 0.919 | 130% |
| | (84/84) | (84/84) | (78/84) | (48/84) | (36/84) | (17/84) | | |
| | c/w > 3 | c/w > 3 | c/w = 2.64 | c/w = 0.85 | c/w = 0.56 | c/w = 0.23 | | |
| c-Met | 24.24 | 27.24 | 28.48 | 29.77 | 29.81 | 29.96 | 0.918 | 133% |
| | (84/84) | (84/84) | (78/84) | (49/84) | (37/84) | (19/84) | | |
| | c/w > 3 | c/w > 3 | c/w = 2.64 | c/w = 0.88 | c/w = 0.58 | c/w = 0.26 | | |

c/w: the average copy number of each reaction well

As shown in Table 2 above, the gene expression amount of Beta-Actin is higher, the correlation coefficient R² is 0.999, and the PCR efficiency is 81%. At the same time, the average copy number of each reaction well is not less than 1, and the Cq value shows a linear relationship, so adjustment is not needed. In comparison, the Cq values of HER2, PD-1, and c-Met in the later stage of a 4-time dilution converge to be constants, the average copy number of each reaction well is less than 1, the correlation coefficient R² is low, and PCR efficiency is poor, so adjustment needs to be performed via the mechanism of the invention.

TABLE 3

| Corrected | | | | | | | Correlation | PCR |
|---|---|---|---|---|---|---|---|---|
| Cq value | 300 ng | 75 ng | 18.75 ng | 9.38 ng | 4.69 ng | 2.34 ng | coefficient R² | efficiency |
| Beta-Actin | 18.39 | 20.94 | 23.28 | 24.43 | 25.52 | 26.62 | 0.999 | 81% |
| HER2 | 26.11 | 28.42 | 30.52 | 31.69 | 33.04 | 34.32 | 0.998 | 82% |
| PD-1 | 24.44 | 27.35 | 28.66 | 30.29 | 30.92 | 32.21 | 0.986 | 91% |
| c-Met | 24.24 | 27.24 | 28.48 | 29.96 | 30.57 | 31.85 | 0.983 | 95% |

TABLE 4

| Corrected | | | | | | | Correlation | PCR |
|---|---|---|---|---|---|---|---|---|
| Cq value | 300 ng | 75 ng | 18.75 ng | 9.38 ng | 4.69 ng | 2.34 ng | coefficient R² | efficiency |
| Beta-Actin | 18.39 | 20.94 | 23.28 | 24.43 | 25.52 | 26.62 | 0.999 | 81% |
| HER2 | 26.11 | 28.42 | 30.69 | 32.72 | 34.17 | 35.54 | 0.992 | 66% |
| PD-1 | 24.44 | 27.35 | 28.66 | 31.11 | 31.70 | 32.76 | 0.980 | 86% |
| c-Met | 24.24 | 27.24 | 28.48 | 30.03 | 30.86 | 32.60 | 0.983 | 86% |

As shown in Table 3 and Table 4 above, after an adjustment step is performed for HER2, PD-1, and c-Met to correct the original Cq value, the correlation coefficient R² can be increased to 0.98 or more. At the same time, the Cq value converges to be a constant can also be adjusted back to the linear relationship, so as to increase the sensitivity and accuracy of qPCR.

Based on the above, the invention provides a measuring method for a nucleic acid sample, which corrects the Cq value using experimental information of qPCR, so as to increase the sensitivity and accuracy of qPCR and extend the dynamic range. Moreover, the drawback of detection accuracy issue for a low concentration range nucleic acid sample in the prior art is alleviated. The Cq value of nucleic acid targets having lower concentrations can be adjusted by using the invention, such that the Cq value converges to be a constant can be corrected back to a linear relationship, so as to extend the detection dynamic linear range. Therefore, the measuring method of the invention is suitable for samples which contain different nucleic acid targets having a variety of concentration ranges, especially for clinical samples.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A measuring method for a nucleic acid sample, comprising:
providing a test plate having a plurality of reaction wells to perform a qPCR reaction on the nucleic acid sample; and
performing an adjustment step according to a positive well measurement value obtained by a number of positive reaction wells to correct an original Cq value.

2. The measuring method for the nucleic acid sample of claim 1, wherein the adjustment step comprises:
drawing a graph by using a nucleic acid template concentration as a horizontal axis and the original Cq value as a vertical axis and obtaining a slope; and
multiplying the slope by a logarithm of positive well measurement value and then adding the original Cq value to obtain a corrected Cq value,
wherein the slope is related to a PCR efficiency.

3. The measuring method for the nucleic acid sample of claim 1, wherein the nucleic acid sample contains more than one kind of nucleic acid target, and the nucleic acid targets have different concentration ranges.

4. The measuring method for the nucleic acid sample of claim 1, wherein the qPCR reaction is performed using 64 reaction wells of the test plate.

5. The measuring method for the nucleic acid sample of claim 4, wherein after using the 64 reaction wells of the test plate and the adjustment step, a dynamic range is increased to 9 logs.

6. The measuring method for the nucleic acid sample of claim 2, wherein a correlation coefficient R² between the nucleic acid template concentration and the corrected Cq value is 0.98 or more.

7. The measuring method for the nucleic acid sample of claim 1, wherein the number of positive reaction wells is divided by a number of all reaction wells to obtain a ratio of positive reaction wells to all reaction wells, and then the ratio is plugged into a Poisson distribution to obtain an average sample copy number of each reaction well, the average sample copy number of each reaction well is the positive well measurement value.

8. The measuring method for the nucleic acid sample of claim 7, wherein when the average sample copy number of each reaction well is less than 1, the adjustment step is performed to correct the original Cq value.

9. The measuring method for the nucleic acid sample of claim 1, wherein the positive well measurement value is a ratio obtained by dividing the number of positive reaction wells by a number of all reaction wells.

10. The measuring method for the nucleic acid sample of claim 9, wherein when the ratio obtained by dividing the number of positive reaction wells by the number of all reaction wells is less than 95%, the adjustment step is performed to correct the original Cq value.
